(19) United States
(12) Patent Application Publication
(10) Pub. No.: US 2025/0261973 A1
(43) Pub. Date: Aug. 21, 2025

(54) PEDICLE SCREW ASSEMBLY

(71) Applicant: Ulrich Medical, USA, Plano, TX (US)

(72) Inventors: Jesse Jordan Hart Dewey, Johnson City, NY (US); Eric Montgomery Lucas, McKinney, TX (US)

(21) Appl. No.: 19/041,205

(22) Filed: Jan. 30, 2025

Related U.S. Application Data
(60) Provisional application No. 63/555,732, filed on Feb. 20, 2024.

(51) Int. Cl. A61B 17/70

(52) U.S. Cl. CPC A61B 17/7034

(57) ABSTRACT

A pedicle screw assembly includes a tulip having a pocket that receives a head of the screw body. The pedicle screw assembly includes a tulip extension that extends from the tulip including first and second extension tabs extending from first and second side walls of the tulip. The tulip extension has a lumen defined between the first and second extensions and slots defined between the first and second extensions aligned with rod openings of the tulip. The tulip extension includes a deflectable retention spring along at least one of the extension tabs. A support ring is coupled to the extension tabs for support. The retention spring retains the support ring on the extension tabs but is releasable to allow removal of the support ring.

### CROSS REFERENCE TO RELATED APPLICATIONS

This application is a claims priority to U.S. Application No. 63/555,732, filed 20 Feb. 2024, the subject matter of which is herein incorporated by reference in its entirety.

### BACKGROUND OF THE INVENTION

The subject matter herein relates generally to pedicle screw assemblies.

A technique commonly referred to as spinal fixation is employed for fusing together and/or mechanically immobilizing vertebrae of the spine. In spine stabilization procedures, connecting elements, such as rods, plates or wires are placed and fixed between two or more locations of the spine. In some applications, pedicle screws can be inserted into the vertebrae of the spine and connected with the connecting elements to provide immobilization and stabilization of the vertebral column. The pedicle screws used in some surgical procedures utilize tubes or extended tabs extending from the ends of the pedicle screws to access the pedicle screws at locations deep in the patient's body. The extensions provide a slot or channel for insertion of the connecting elements or tools to access the pedicle screw. However, fixation of the extensions may be difficult. If the extensions collapse inward, it is difficult or impossible to load the connecting element or tool into the pedicle screw. If the extensions spread outward, it may be difficult to properly load the connecting element or tool into the pedicle screw. Additionally, opening or closing of the extensions may cause the extensions to be damaged or separate from the pedicle screw.

### BRIEF DESCRIPTION OF THE INVENTION

In one embodiment, a pedicle screw assembly is provided and includes a screw body that has a head and a threaded shaft extends from the head. The pedicle screw assembly includes a tulip that has a pocket that receives the head of the screw body. The tulip has a first side wall and a second side wall on opposite sides of the pocket. The tulip has rod openings defined between the first and second side walls configured to receive a rod. The pedicle screw assembly includes a set screw received in the pocket to secure the rod in the slot. The pedicle screw assembly includes a tulip extension that extends from the tulip. The tulip extension includes a first extension tab that extends from the first side wall and a second extension tab extends from the second side wall. The first and second side walls extend parallel to each other between proximal ends and distal ends of the first and second extension tabs. The tulip extension has a lumen defined between the first and second extensions. The tulip extension has slots defined between the first and second extensions aligned with the rod openings. The tulip extension includes a deflectable retention spring along the first extension tab. The pedicle screw assembly includes a support ring coupled to the distal ends of the first and second extension tabs to support the first and second extension tabs relative to each other. The support ring interfaces with the retention spring to retain the support ring on the first and second extension tabs. The retention spring is releasable from the support ring to allow removal of the support ring from the distal ends of the first and second extension tabs.

In another embodiment, a pedicle screw assembly is provided and includes a screw body that has a head and a threaded shaft extends from the head. The pedicle screw assembly includes a tulip that has a pocket to receive the head of the screw body. The tulip has a first side wall and a second side wall on opposite sides of the pocket. The tulip has rod openings defined between the first and second side walls configured to receive a rod. The pedicle screw assembly includes a set screw received in the pocket to secure the rod in the slot. The pedicle screw assembly includes a tulip extension that extends from the tulip. The tulip extension includes a first extension tab that extends from the first side wall and a second extension tab extends from the second side wall. The first and second side walls extend parallel to each other between proximal ends and distal ends of the first and second extension tabs. The tulip extension has a lumen defined between the first and second extensions. The tulip extension has slots defined between the first and second extensions aligned with the rod openings. The pedicle screw assembly includes a support ring that includes a ring body surrounding an opening configured to receive the distal ends of the first and second extension tabs. The ring body surrounds the first and second extension tabs to prevent opening of the first and second extension tabs relative to each other. The ring body is coupled to the first and second extension tabs to support the first and second extension tabs and prevent closing of the first and second extension tabs relative to each other.

In a further embodiment, a pedicle screw assembly is provided and includes a screw body that has a head and a threaded shaft extends from the head. The pedicle screw assembly includes a tulip that has a pocket to receive the head of the screw body. The tulip has a first side wall and a second side wall on opposite sides of the pocket. The tulip has rod openings defined between the first and second side walls configured to receive a rod. The pedicle screw assembly includes a set screw received in the pocket to secure the rod in the slot. The pedicle screw assembly includes a tulip extension extends from the tulip. The tulip extension includes a first extension tab that extends from the first side wall and a second extension tab extends from the second side wall. The first and second side walls extend parallel to each other between proximal ends and distal ends of the first and second extension tabs. The tulip extension has a lumen defined between the first and second extensions. The tulip extension has slots defined between the first and second extensions aligned with the rod openings. The tulip extension includes a deflectable retention spring along the first extension tab. The pedicle screw assembly includes a support ring that includes a ring body surrounding an opening configured to receive the distal ends of the first and second extension tabs. The ring body surrounds the first and second extension tabs to prevent opening of the first and second extension tabs relative to each other. The ring body is coupled to the first and second extension tabs to support the first and second extension tabs and prevent closing of the first and second extension tabs relative to each other. The support ring interfaces with the retention spring to retain the support ring on the first and second extension tabs. The retention spring is releasable from the support ring to allow removal of the support ring from the distal ends of the first and second extension tabs.

### BRIEF DESCRIPTION OF THE DRAWINGS

FIG. 1 is a front perspective view of a pedicle screw assembly in accordance with an exemplary embodiment.

FIG. 2 is a rear perspective view of the pedicle screw assembly in accordance with an exemplary embodiment.

FIG. 3 is a front, exploded perspective view of the pedicle screw assembly in accordance with an exemplary embodiment.

FIG. 4 is a side, exploded perspective view of the pedicle screw assembly in accordance with an exemplary embodiment.

FIG. 5 is a bottom perspective view of the support ring in accordance with an exemplary embodiment.

FIG. 6 is a top perspective view of the support ring in accordance with an exemplary embodiment.

FIG. 7 is a top view of the support ring in accordance with an exemplary embodiment.

FIG. 8 is a side view of a portion of the pedicle screw assembly showing the tulip extension in accordance with an exemplary embodiment.

FIG. 9 is a front view of a portion of the pedicle screw assembly showing the tulip extension in accordance with an exemplary embodiment.

FIG. 10 is a perspective view of a portion of the pedicle screw assembly showing the tulip extension in accordance with an exemplary embodiment.

FIG. 11 is a side view of a portion of the pedicle screw assembly showing the top of the tulip extension in accordance with an exemplary embodiment.

FIG. 12 is a front view of a portion of the pedicle screw assembly showing the top of the tulip extension in accordance with an exemplary embodiment.

FIG. 13 is an exploded, perspective view of a portion of the pedicle screw assembly showing the support ring poised for loading onto the tulip extension in accordance with an exemplary embodiment.

FIG. 14 is a perspective view of a portion of the pedicle screw assembly showing the support ring coupled to the tulip extension in accordance with an exemplary embodiment.

FIG. 15 is a side view of a portion of the pedicle screw assembly showing the support ring on the tulip extension in accordance with an exemplary embodiment.

FIG. 16 is a cross-sectional view of a portion of the pedicle screw assembly showing the support ring on the tulip extension in accordance with an exemplary embodiment.

FIG. 17 is a perspective, sectional view of a portion of the pedicle screw assembly showing the support ring on the tulip extension in accordance with an exemplary embodiment.

FIG. 18 is a cross-sectional view of the pedicle screw assembly in accordance with an exemplary embodiment.

FIG. 19 is a cross-sectional view of the pedicle screw assembly through the support ring facing downward in accordance with an exemplary embodiment.

FIG. 20 is a cross-sectional view of the pedicle screw assembly through the support ring facing upward in accordance with an exemplary embodiment.

### DETAILED DESCRIPTION OF THE INVENTION

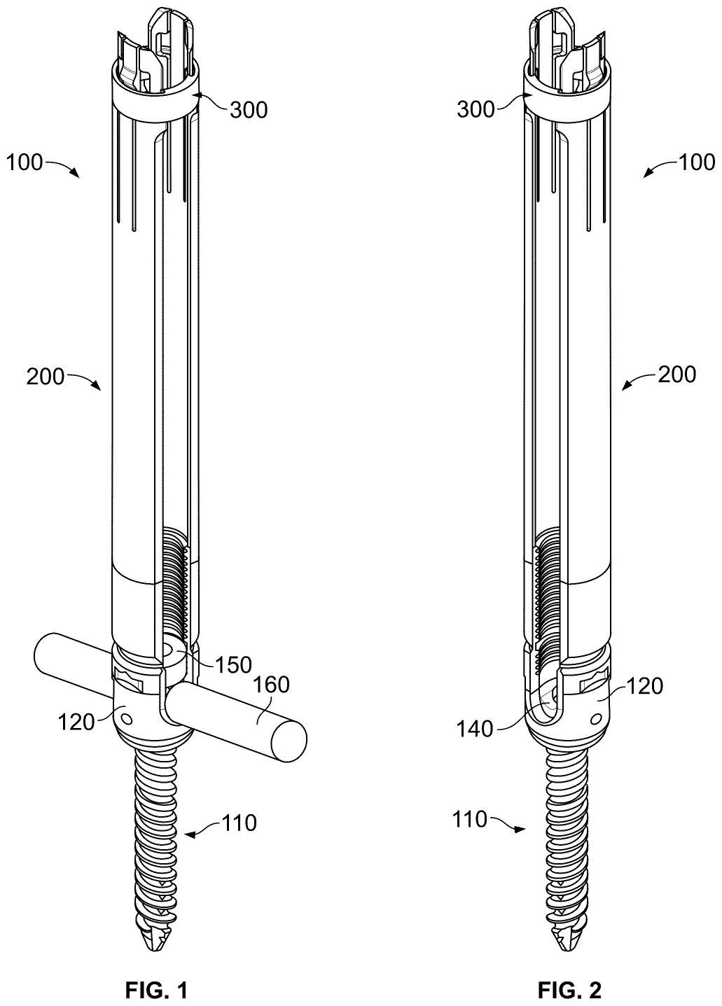

FIG. 1 is a front perspective view of a pedicle screw assembly 100 in accordance with an exemplary embodiment. FIG. 2 is a rear perspective view of the pedicle screw assembly 100 in accordance with an exemplary embodiment. The pedicle screw assembly 100 includes a screw body 110, a tulip 120, a clamping plate 140 and a set screw 150 (FIG. 1) used to secure a connecting element 160 (FIG. 1), such as a rod, in the tulip 120. The pedicle screw assembly 100 includes a tulip extension 200 extending from the tulip 120 and a support ring 300 used to support the tulip extension 200.

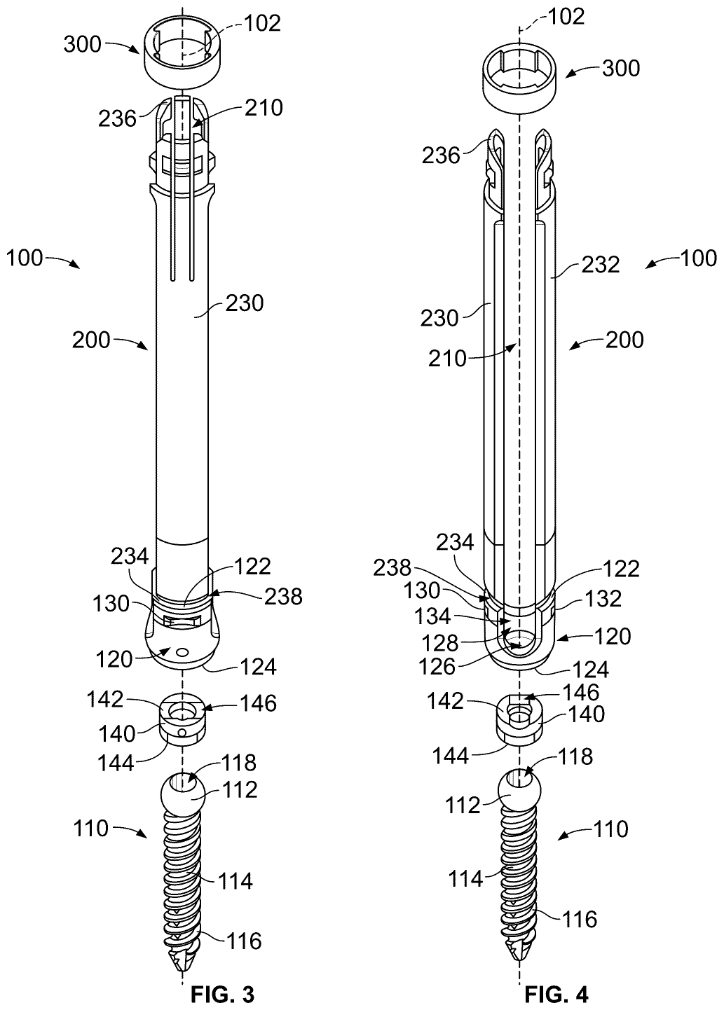

FIG. 3 is a front, exploded perspective view of the pedicle screw assembly 100 in accordance with an exemplary embodiment. FIG. 4 is a side, exploded perspective view of the pedicle screw assembly 100 in accordance with an exemplary embodiment. FIGS. 3 and 4 illustrate the screw body 110, the tulip 120, the clamping plate 140, the tulip extension 200, and the support ring 300 axially aligned along a longitudinal axis 102 of the pedicle screw assembly 100.

The screw body 110 includes a head 112 and a threaded shaft 114 extending from the head 112. The threaded shaft 114 includes helical threads 116 around the exterior of the threaded shaft 114. The threaded shaft 114 is configured to be screwed into a bone of the patient. In an exemplary embodiment, the head 112 includes a drive element 118 is configured to interface with a drive tool to screw the threaded shaft 114 into the bone of the patient. In the illustrated embodiment, the drive element 118 is an opening, such as a hexagonal opening, configured to receive an end of a drive tool. In the illustrated embodiment, the head 112 is spherical to allow multiaxial orientation of the screw body 110 relative to the tulip 120. The clamping plate 140 is used to secure the axial orientation of the screw body 110 relative to the tulip 120, such as in a direction that is nonparallel to the longitudinal axis 102.

The clamping plate 140 includes an upper surface 142 and a lower surface 144. The lower surface 144 is configured to engage the head 112 of the screw body 110 to fix the orientation of the screw body 110 relative to the tulip 120. The lower surface 144 may include a concave pocket that receives the head 112. In an exemplary embodiment, the upper surface 142 includes a pocket 146 that is configured to receive the connecting element 160 (FIG. 1).

The tulip 120 extends between a top 122 and a bottom 124. The tulip 120 has a cavity 126 between the top 122 and the bottom 124. The cavity 126 receives the screw body 110, the clamping plate 140, and the set screw 150 (FIG. 1). In an exemplary embodiment, the tulip 120 includes a pocket 128 at the bottom of the cavity 126. The pocket 128 receives the head 112 of the screw body 110. The pocket 128 may have a complementary shape as the head 112 to allow multiaxial rotation of the head 112 within the pocket 128.

In an exemplary embodiment, the tulip 120 includes a first side wall 130 and a second side wall 132 on opposite sides of the cavity 126. The first and second side walls 130, 132 extend to the top 122 of the tulip 120. In various embodiments, the first and second side walls 130, 132 extend generally parallel to the longitudinal axis 102. The first and second side walls 130, 132 are curved or arcuate shape, such as forming portions of the circumference of the tulip 120. In an exemplary embodiment, the tulip extension 200 extends from the first and second side walls 130, 132. The tulip extension 200 is removable and/or separable from the first and second side walls 130, 132 after the pedicle screw assembly 100 is fixed to the patient.

The connecting element/rod 160 (FIG. 1) is configured be received in the cavity 126 between the first and second side walls 130, 132. The tulip 120 includes rod openings 134 at opposite sides of the tulip 120. The rod openings 134 are open between the first and second side walls 130, 132. In the illustrated embodiment, the rod openings 134 are U-shaped. However, the rod openings 134 may have other shapes in alternative embodiments.

The tulip extension 200 extends from the tulip 120. The tulip extension 200 forms a lumen 210 aligned with the cavity 126 of the tulip 120. The lumen 210 is used to guide components into the cavity 126. For example, when the tulip 120 is located within the patient's body, the tulip extension 200 may extend through an incision to and an exterior of the patient's body. The components may be loaded into the patient's body through the lumen 210. For example, the screw body 110 and/or the clamping plate 140 and/or the rod 160 and/or the set screw 150 may be loaded into the cavity 126 through the lumen 210. Tools or other components may additionally be loaded into the cavity 126 through the lumen 210 during the procedure. The tulip extension 200 guides the components and/or the tools into the cavity 126 of the tulip 120.

In an exemplary embodiment, the tulip extension 200 includes a first extension tab 230 extending from the first side wall 130 and a second extension tab 232 extending from the second side wall 132. The first and second extensions tabs 230, 232 extend parallel to each other, such as parallel to the longitudinal axis 102. Optionally, the extensions tabs 230, 232 may be similar to each other, and like components identified with like reference numerals. In various embodiments, the extensions tabs 230, 232 are identical to each other being oriented on opposite sides of the tulip extension 200, such as being mirrored halves. In an exemplary embodiment, each extension tab 230, 232 extends between a proximal end 234 and a distal end 236. The proximal end 234 of the each extension tab 230, 232 is connected to the top 122 of the corresponding side wall 130, 132. Optionally, a score line or cut line 238 is provided between the extension tab 230, 232 and the corresponding side wall 130, 132 to allow easy separation of the tulip extension 200 from the tulip 120 after the pedicle screw assembly 100 is fixed to the patient's bone. The score line 238 may be provided at the exterior surfaces and/or the interior surfaces.

The support ring 300 is configured to be coupled to the tulip extension 200, such as at the distal ends 236 of the extensions tabs 230, 232. The support ring 300 is used to hold relative positions of the extensions tabs 230, 232. When the support ring 300 is coupled to the tulip extension 200, the support ring 300 surrounds the first and second extensions tabs 230, 232 to prevent opening of the first and second extensions tabs 230, 232 relative to each other. For example, the support ring 300 prevents spreading apart of the extensions tabs 230, 232. The support ring 300 supports the first and second extensions tabs 230, 232 to prevent closing of the first and second extensions tabs 230, 232 relative to each other. For example, the support ring 300 prevents moving the extensions tabs 230, 232 toward each other. The support ring 300 holds the extensions tabs 230, 232 parallel to each other to maintain the relative dimension of the lumen 210 along the tulip extension 200. In an exemplary embodiment, the tulip extension 200 includes one or more features to position the support ring 300 on the extensions tabs 230, 232. In an exemplary embodiment, the tube extensions 200 includes one or more retention features to retain the support ring 300 on the tulip extension 200. In an exemplary embodiment, the retention features are releasable to allow removal of the support ring 300, such as for loading components into the lumen 210 and/or to allow separation of the tulip extension 200 from the tulip 120.

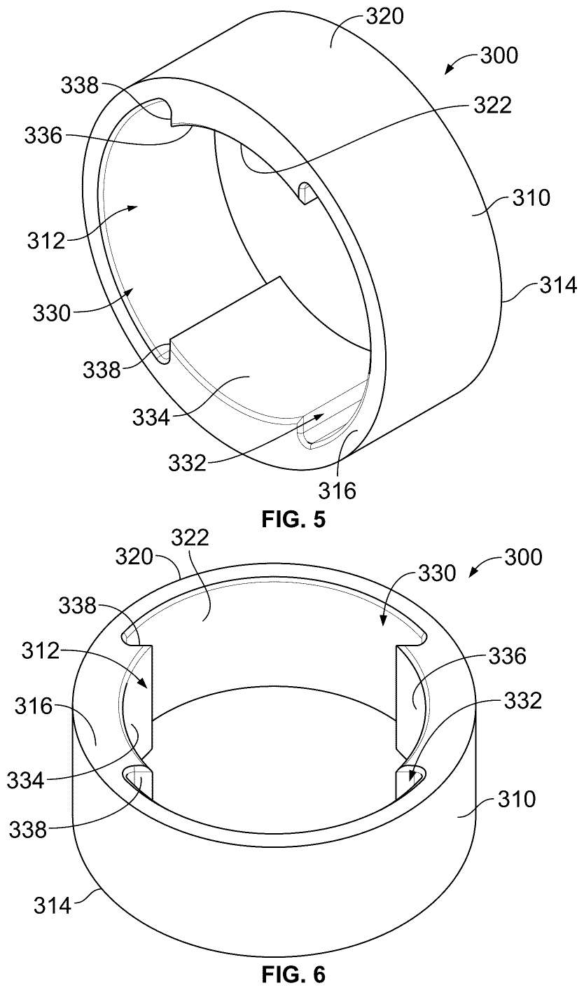

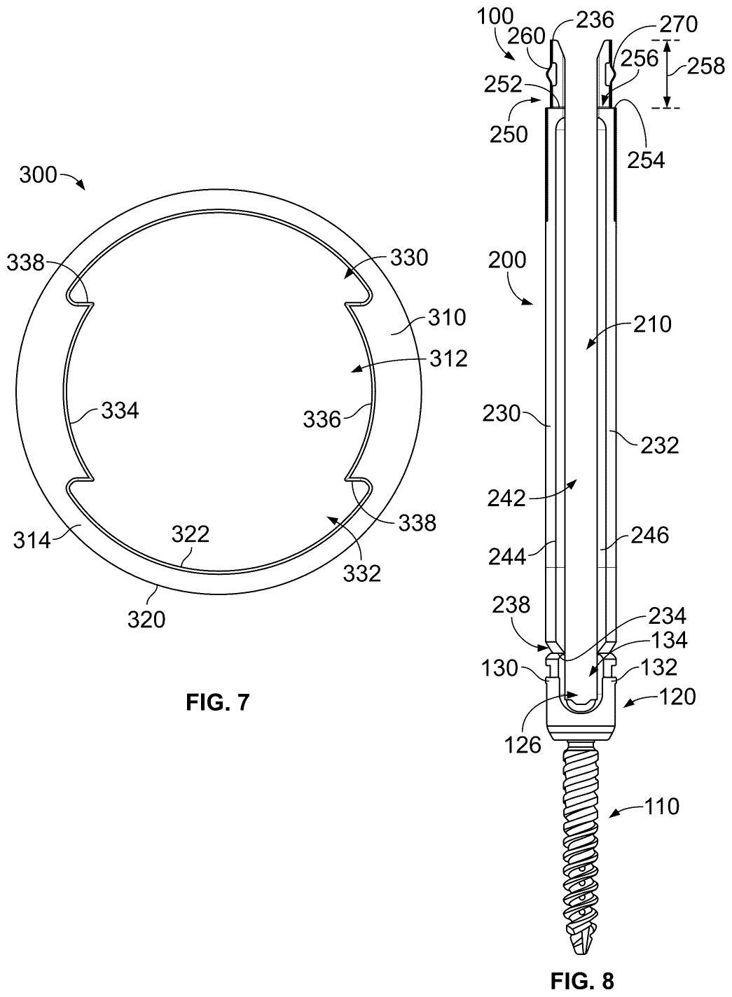

FIG. 5 is a bottom perspective view of the support ring 300 in accordance with an exemplary embodiment. FIG. 6 is a top perspective view of the support ring 300 in accordance with an exemplary embodiment. FIG. 7 is a top view of the support ring 300 in accordance with an exemplary embodiment.

The support ring 300 includes a ring body 310 surrounding an opening 312. The support ring 300 extends between a top 314 and a bottom 316. Optionally, the top 314 and/or the bottom 316 may be planar. In various embodiments, the top 314 and the bottom 316 are parallel to each other. The support ring 300 includes an exterior surface 320 extending between the top 314 and the bottom 316. In an exemplary embodiment, the exterior surface 320 is cylindrical. The support ring 300 has an outer diameter defined by the exterior surface 320. The support ring 300 includes an interior surface 322 surrounding the opening 312. The support ring 300 has an inner diameter defined by the interior surface 322.

In an exemplary embodiment, the support ring 300 includes a first tab channel 330 along a first side of the opening 312 and a second tab channel 332 along a second side of the opening. The tab channels 330, 332 are open at the interior surface 322 to the opening 312. The tab channels 330, 332 are configured to receive the corresponding extensions tabs 230, 232 (FIG. 3). In an exemplary embodiment, the support ring 300 includes support pads 334, 336 on opposite sides of the opening 312. The support pads 334, 336 are located between the tab channels 330, 332. The support pads 334, 336 are used to support the extensions tabs 230, 232. In an exemplary embodiment, the support pads 334, 336 include tab hooks 338 extending along the tab channels 330, 332. The tab hooks 338 surrounding pockets that receive portions of the extensions tabs 230, 232 to interface with the extensions tabs 230, 232 and hold the extensions tabs 230, 232 in the tab channels 330, 332. The opening 312 has a smaller diameter between the support pads 334, 336 compared to the tab channels 330, 332. The opening 312 has a larger diameter between the tab channels 330, 332 compared to the support pads 334, 336.

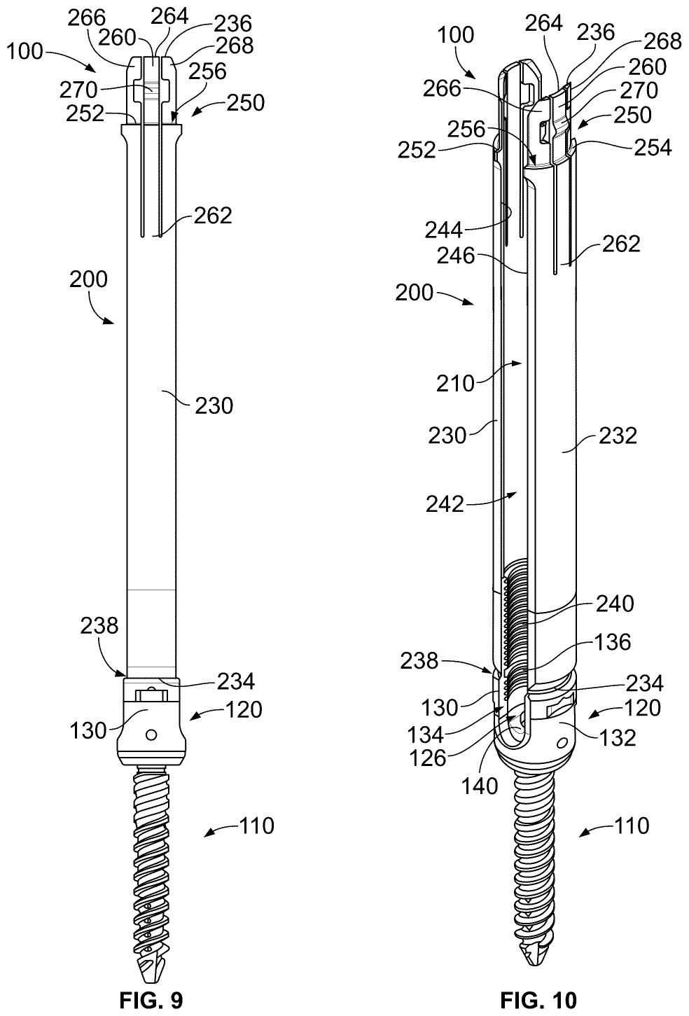

FIG. 8 is a side view of a portion of the pedicle screw assembly 100 showing the tulip extension 200 in accordance with an exemplary embodiment. FIG. 9 is a front view of a portion of the pedicle screw assembly 100 showing the tulip extension 200 in accordance with an exemplary embodiment. FIG. 10 is a perspective view of a portion of the pedicle screw assembly 100 showing the tulip extension 200 in accordance with an exemplary embodiment. The tulip extension 200 extends from the top of the tulip 120. The screw body 110 and the clamp a plate 140 are shown in FIGS. 8-10.

The tulip extension 200 includes the first and second extensions tabs 230, 232 extending from the first and second side walls 130, 132 of the tulip 120. The lumen 210 is defined between the first and second extensions tabs 230, 232. The extensions tabs 230, 232 extend between the proximal ends 234 and the distal ends 236. The score lines 238 are provided at the proximal ends 234. In an exemplary embodiment, the side walls 130, 132 of the tulip 120 include internal threads 136 configured to threadably receive the set screws 150 (FIG. 1). In an exemplary embodiment, the proximal ends 234 of the extensions tabs 230, 232 include internal threads 240. The internal threads 240 aligned with the internal threads 136 to form a continuous threaded portion that receives the set screws 150. The internal threads 240, 136 may receive other components, tools, or devices.

In an exemplary embodiment, the first and second extensions tabs 230, 232 are separated from each other by slots 242. The slots 242 are formed between edges 244, 246 of the extensions tabs 230, 232. The slot 242 are arranged on opposite sides of the tulip extension 200. In an exemplary embodiment, the slots 242 extend the entire height of the tulip extension 200. For example, the slots 242 are open at the top of the tulip extension 200, such as to receive the rod 160 (FIG. 1) or other tools. The slots 242 are aligned with the rod openings 134 to allow the rod 160 to pass from the lumen 210 of the tulip extension 200 into the cavity 126 of the tulip 120.

In an exemplary embodiment, the tulip extension 200 includes a collar 250 at the top of the tulip extension 200 that is configured to receive the support ring 300 (FIG. 5). In an exemplary embodiment, the tulip extension 200 has a reduced diameter at the collar 250 to receive the support ring 300. The collar 250 includes a first shoulder 252 along the first extension tab 230 and a second shoulder 254 along the second extension tab 232. The first and second shoulders 252, 254 form a seat 256 for the support ring 300 to locate the support ring 300 along the first and second extensions tabs 230, 232. The seat 256 is located a distance 258 from the distal ends 236 of the extensions tabs 230, 232. The seat 256 is located near the top of the tulip extension 200 and is located remote from the tulip 120.

In an exemplary embodiment, the tulip extension 200 includes one or more retention springs 260 for retaining the support ring 300 on the tulip extension 200. In the illustrated embodiment, both extensions tabs 230, 232 include one of the retention springs 260. Greater or fewer retention springs 260 may be provided in alternative embodiments. In an exemplary embodiment, the retention springs 260 are deflectable relative to the extensions tabs 230, 232 to release the retention springs 260 from the support ring 300 and allow removal of the support ring 300 from the tulip extension 200. For example, the retention springs 260 may be flexed or deflected inward into the lumen 210 to release the retention springs 260 from the support ring 300 to allow removal of the support ring 300 from the tulip extension 200.

In an exemplary embodiment, the retention springs 260 are integral with the extensions tabs 230, 232. For example, the retention springs 260 may be stamped or cut from the extensions tabs 230, 232. For example, parallel cut lines may be formed on both sides of the retention spring 260 to release the retention spring 260 from the extension tab 230, 232 and allow movement of the retention spring 260 relative to the extension tab 230, 232. Each retention spring 260 extends between a fixed end 262 and a free end 264. The retention spring 260 is cantilevered from the fixed end 262 the free end 264 is movable relative to the corresponding extension tab 230, 232. In an exemplary embodiment, the retention spring 260 is provided at the top of the tulip extension 200. For example, the free end 264 may be located at the distal end 236 of the extension tab 230, 232. In an exemplary embodiment, the extension tab 230, 232 includes a flanking portions 266, 268 along both sides of the retention spring 260. The retention spring 260 is movable relative to the flanking portions 266, 268. In alternative embodiments, the extension tab 230, 232 may have only one of the flanking portions 266 or 268 along only one side of the retention spring 260. In an exemplary embodiment, the retention spring 260 has a complementary shape as the flanking portions 266, 268 such that the retention spring 260 forms part of the profile of the extension tab 230, 232. In an exemplary embodiment, the retention spring 260 includes a portion of the seat 256.

In an exemplary embodiment, the retention spring 260 includes a lip 270 protruding outwardly therefrom to engage the support ring 300. The lip 270 is a retaining feature of the retention spring 260. The lip 270 is spaced apart from the seat 256 to form a space therebetween that receives and captures the support ring 300. When the retention spring 260 is released and flexed inward, the lip 270 is configured to be released from the support ring 300 to allow removal of the support ring 300 from the tulip extension 200.

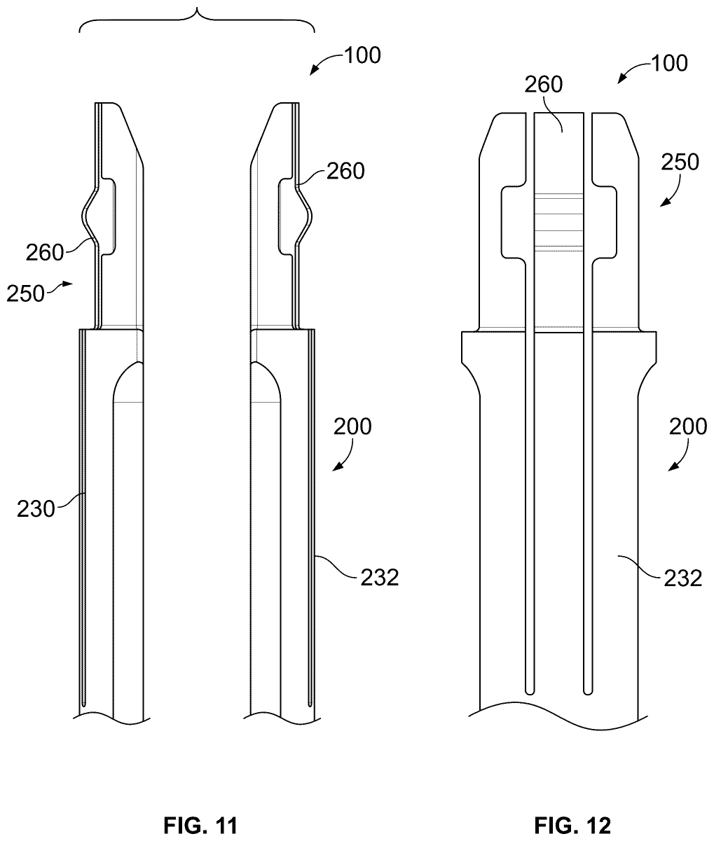

FIG. 11 is a side view of a portion of the pedicle screw assembly 100 showing the top of the tulip extension 200 in accordance with an exemplary embodiment. FIG. 12 is a front view of a portion of the pedicle screw assembly 100 showing the top of the tulip extension 200 in accordance with an exemplary embodiment. FIGS. 11 and 12 illustrate the collar 250 at the top of the tulip extension 200 that is configured to receive the support ring 300 (FIG. 5). FIGS. 11 and 12 illustrate the retention springs 260 formed in the extensions tabs 230, 232. The tulip extension 200 may include other shapes or features to retain and locate the support ring 300 at the top of the tulip extension 200 in alternative embodiments.

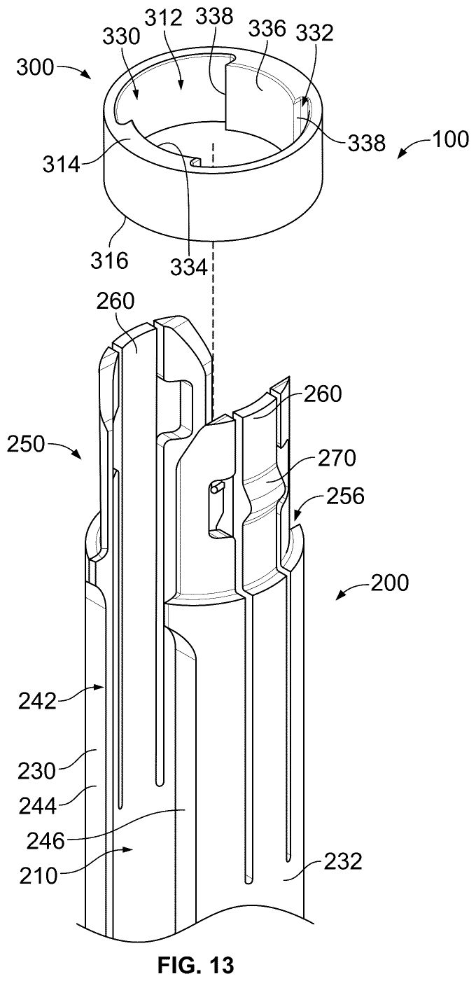

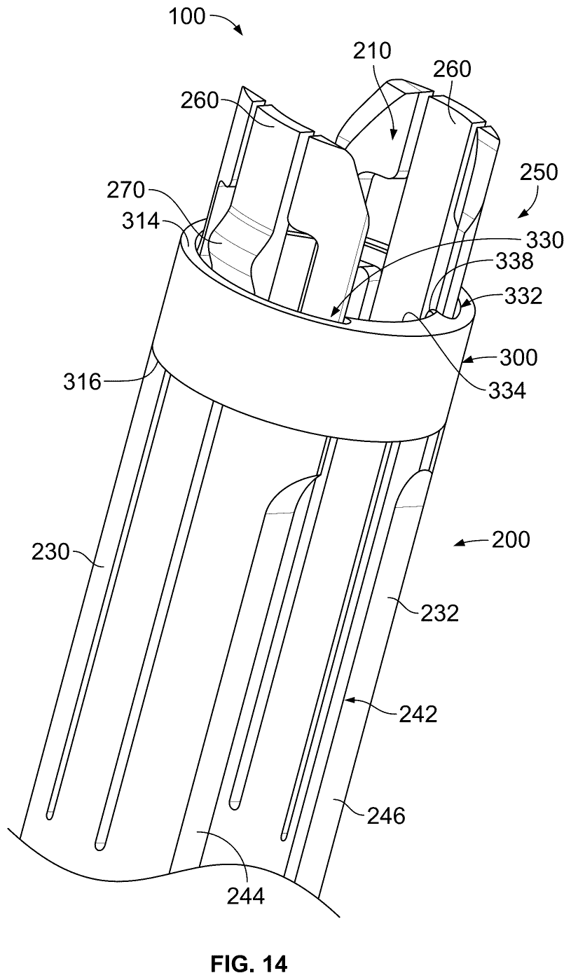

FIG. 13 is an exploded, perspective view of a portion of the pedicle screw assembly 100 showing the support ring 300 poised for loading onto the tulip extension 200 in accordance with an exemplary embodiment. FIG. 14 is a perspective view of a portion of the pedicle screw assembly 100 showing the support ring 300 coupled to the tulip extension 200 in accordance with an exemplary embodiment.

During assembly, the support ring 300 is aligned with the top of the tulip extension 200. The tulip extension 200 is received in the opening 312. The support ring 300 is loaded onto the tulip extension 200 until the bottom 316 of the support ring 300 engages the seat 256. The retention springs 260 engage the support ring 300 and retain the support ring 300 on the collar 250. For example, the lips 270 of the retention springs 260 engage the top 314 of the support ring 300 and prevent upward movement of the support ring 300 relative to the tulip extension 200.

In an exemplary embodiment, the first and second extensions tabs 230, 232 are received in the first and second tab channels 330, 332, respectively. When assembled, the support ring 300 circumferentially surrounds the top end of the tulip extension 200 to prevent spreading apart of the extensions tabs 230, 232. The support pads 334, 336 are located in the slots 242 between the extensions tabs 230, 232. The edges 244, 246 of the extensions tabs 230, 232 are received in the pockets of the support pads 334, 336 such that the tab hooks 338 hook partially around the edges 244, 246. The tab hooks 338 engage the extensions tabs 230, 232 to hold the extensions tabs 230, 232 in the tab channels 330, 332. The extensions tabs 230, 232 are captured in the tab channels 330, 332 to prevent inward movement of the extensions tabs 230, 232 relative to each other. The support pads 334, 336 block inward movement of the extensions tabs 230, 232 thus maintaining the parallel orientations of the extensions tabs 230, 232 and preventing closing of the lumen 210. However, the retention springs 260 are unblocked by the support pads 334, 336 and are movable relative to the support ring 300 to release from the support ring 300 and allow removal of the support ring 300 from the tulip extension 200.

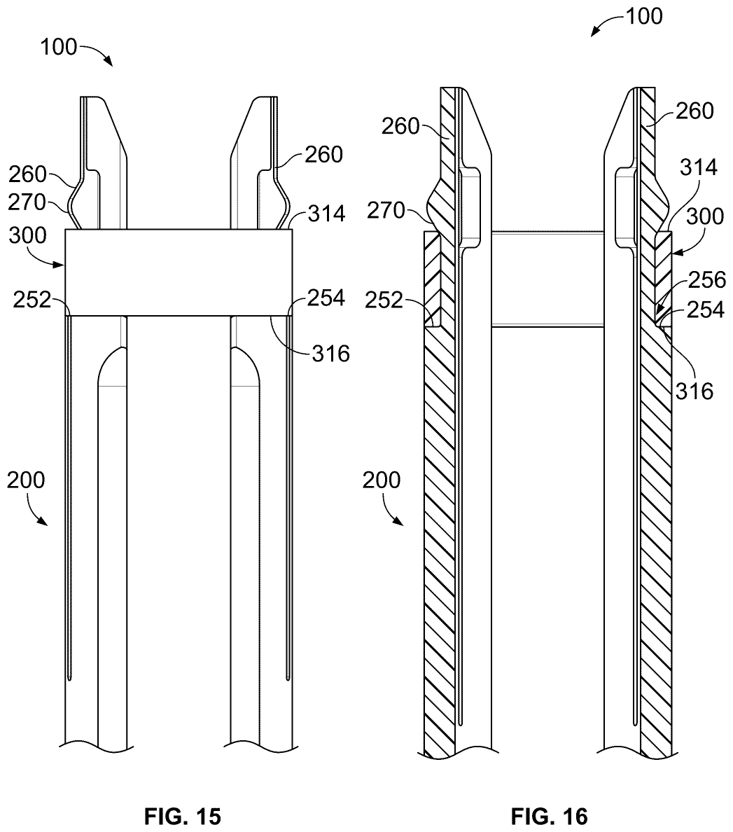

FIG. 15 is a side view of a portion of the pedicle screw assembly 100 showing the support ring 300 on the tulip extension 200. FIG. 16 is a cross-sectional view of a portion of the pedicle screw assembly 100 showing the support ring 300 on the tulip extension 200. When assembled, the bottom 316 of the support ring 300 rests on the shoulders 252, 254 defining the seat 256. The retention springs 260 engage the top 314 of the support ring 300 to hold the support ring 300 on the seat 256. The lip 270 of the retention spring 260 may include an angled ramp surface that faces the support ring 300 and drives the support ring 300 downward against the seat 256.

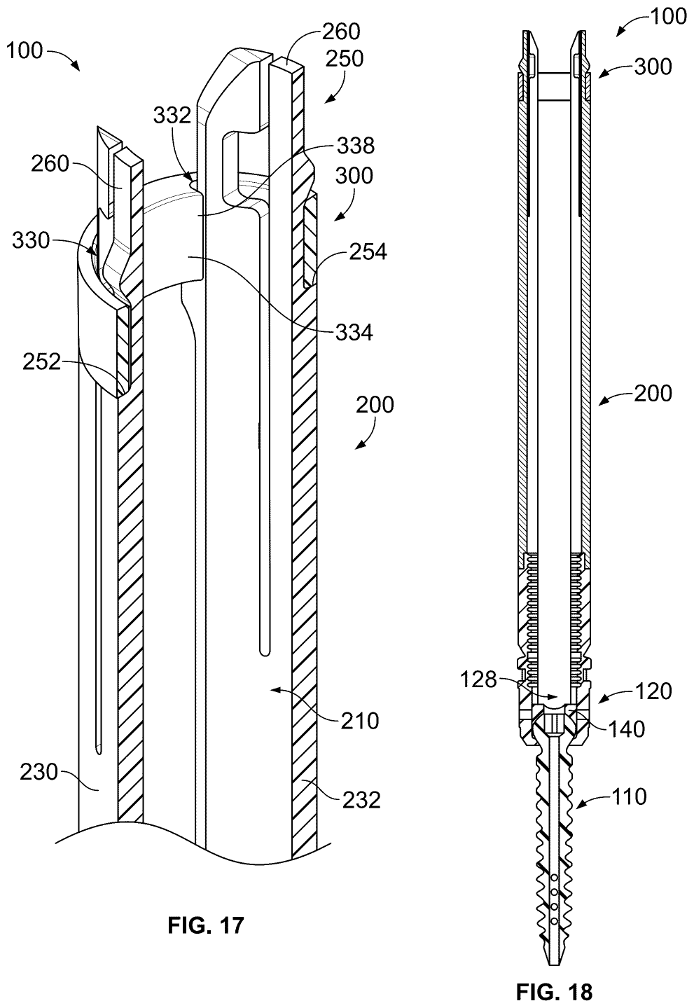

FIG. 17 is a perspective, sectional view of a portion of the pedicle screw assembly 100 showing the support ring 300 on the tulip extension 200. The support ring 300 is shown seated on the shoulders 252, 254 and captured on the collar 250 by the retention springs 260. The support ring 300 surrounds the exterior surfaces of the extensions tabs 230, 232 to prevent outward movement of the extensions tabs 230, 232 relative to each other. FIG. 17 further shows the extensions tabs 230, 232 received in the tab channels 330, 332 of the support ring 300. The support pad 334 is shown between the extensions tabs 230, 232. The tab hooks 338 capture the extensions tabs 230, 232 in the tab channels 330, 332 and prevent inward movement of the extensions tabs 230, 232 relative to each other. As such, the support ring 300 provides both inward support and outward support for the extensions tabs 230, 232 to maintain relative positions of the extensions tabs 230, 232 and maintain the shape of the lumen 210.

FIG. 18 is a cross-sectional view of the pedicle screw assembly 100 in accordance with an exemplary embodiment. FIG. 18 shows the support ring 300 on the tulip extension 200. FIG. 18 shows the tulip extension 200 extending from the tulip 120. FIG. 18 further shows the screw body 110 extending from the bottom of the tulip 120 and the clamping plate 140 holding the screw body 110 in the pocket 128 at the bottom of the tulip 120.

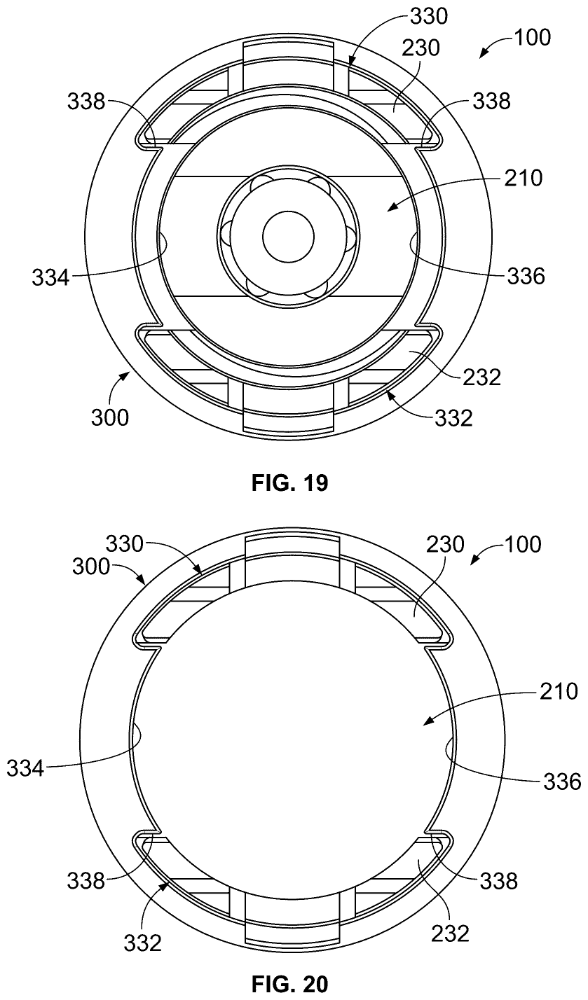

FIG. 19 is a cross-sectional view of the pedicle screw assembly 100 through the support ring 300 facing downward. FIG. 20 is a cross-sectional view of the pedicle screw assembly 100 through the support ring 300 facing upward. The support ring 300 surrounds the exterior surfaces of the extensions tabs 230, 232 to prevent outward movement of the extensions tabs 230, 232 relative to each other. The extensions tabs 230, 232 are received in the tab channels 330, 332 of the support ring 300. The support pads 334, 336 are located between the extensions tabs 230, 232. The tab hooks 338 capture the extensions tabs 230, 232 in the tab channels 330, 332 and prevent inward movement of the extensions tabs 230, 232 relative to each other. As such, the support ring 300 provides both inward support and outward support for the extensions tabs 230, 232 to maintain relative positions of the extensions tabs 230, 232 and maintain the shape of the lumen 210.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112 (f), unless and until such claim limitations expressly use the phrase “means for” followed by a statement of function void of further structure.

What is claimed is:

1. A pedicle screw assembly comprising:
a screw body having a head and a threaded shaft extending from the head;
a tulip having a pocket receiving the head of the screw body, the tulip having a first side wall and a second side wall on opposite sides of the pocket, the tulip having rod openings defined between the first and second side walls configured to receive a rod;
a set screw received in the pocket to secure the rod in the slot;
a tulip extension extending from the tulip, the tulip extension including a first extension tab extending from the first side wall and a second extension tab extending from the second side wall, the first and second side walls extending parallel to each other between proximal ends and distal ends of the first and second extension tabs, the tulip extension having a lumen defined between the first and second extensions, the tulip extension having slots defined between the first and second extensions aligned with the rod openings, the tulip extension including a deflectable retention spring along the first extension tab; and
a support ring coupled to the distal ends of the first and second extension tabs to support the first and second extension tabs relative to each other, wherein the support ring interfaces with the retention spring to retain the support ring on the first and second extension tabs, and wherein the retention spring is releasable from the support ring to allow removal of the support ring from the distal ends of the first and second extension tabs.

2. The pedicle screw assembly of claim 1, wherein the retention spring includes a lip protruding outwardly to engage the support ring.

3. The pedicle screw assembly of claim 1, wherein the retention spring is coincident with the first extension tab.

4. The pedicle screw assembly of claim 1, wherein the retention spring passes through an opening in the support ring along an interior of the support ring.

5. The pedicle screw assembly of claim 1, wherein the support ring includes an interior surface and an exterior surface, the retention spring engaging the exterior surface of the support ring to retain the support ring on the tulip extension, the interior surface engaging the first and second extension tabs.

6. The pedicle screw assembly of claim 1, wherein the retention spring is stamped from the first extension tab.

7. The pedicle screw assembly of claim 1, wherein the retention spring is a first retention spring, the tulip extension further comprises a second retention spring along the second extension tab.

8. The pedicle screw assembly of claim 7, wherein the second retention spring is on an opposite side of the lumen as the first retention spring.

9. The pedicle screw assembly of claim 1, wherein the retention spring is deflectable inward into the lumen to release the support ring.

10. The pedicle screw assembly of claim 1, wherein the tulip extension includes a collar having a first shoulder along the first extension tab and a second shoulder along the second extension tab, the first and second shoulders forming a seat for the support ring to locate the support ring along the first and second extension tabs, the retention spring holding the support ring on the seat.

11. The pedicle screw assembly of claim 1, wherein the first and second tab extensions are removable from the first and second side walls.

12. The pedicle screw assembly of claim 1, wherein the support ring includes a ring body surrounding an opening configured to receive the distal ends of the first and second extension tabs, the ring body surrounding the first and second extension tabs to prevent opening of the first and second extension tabs relative to each other, the ring body coupled to the first and second extension tabs to support the first and second extension tabs and prevent closing of the first and second extension tabs relative to each other.

13. The pedicle screw assembly of claim 12, wherein the ring body includes a first tab channel along a first side of the opening and a second tab channel along a second side of the opening, the first tab channel receiving the first tab extension, the second tab channel receiving the second tab extension.

14. The pedicle screw assembly of claim 13, wherein the ring body includes tab hooks defining the first and second tab channels, the tab hooks capturing the first and second tab extensions in the first and second tab channels to prevent inward movement of the first and second tab extensions.

15. The pedicle screw assembly of claim 12, wherein the ring body includes support pads between the first and second tab channels, the support pads engaging edges of the first and second tab extensions to prevent inward movement of the first and second tab extensions.

16. A pedicle screw assembly comprising:
a screw body having a head and a threaded shaft extending from the head;
a tulip having a pocket receiving the head of the screw body, the tulip having a first side wall and a second side wall on opposite sides of the pocket, the tulip having rod openings defined between the first and second side walls configured to receive a rod;
a set screw received in the pocket to secure the rod in the slot;
a tulip extension extending from the tulip, the tulip extension including a first extension tab extending from the first side wall and a second extension tab extending from the second side wall, the first and second side walls extending parallel to each other between proximal ends and distal ends of the first and second extension tabs, the tulip extension having a lumen defined between the first and second extensions, the tulip extension having slots defined between the first and second extensions aligned with the rod openings; and
a support ring including a ring body surrounding an opening configured to receive the distal ends of the first and second extension tabs, the ring body surrounding the first and second extension tabs to prevent opening of the first and second extension tabs relative to each other, the ring body coupled to the first and second extension tabs to support the first and second extension tabs and prevent closing of the first and second extension tabs relative to each other.

17. The pedicle screw assembly of claim 16, wherein the ring body includes a first tab channel along a first side of the opening and a second tab channel along a second side of the opening, the first tab channel receiving the first tab extension, the second tab channel receiving the second tab extension.

18. The pedicle screw assembly of claim 17, wherein the ring body includes tab hooks defining the first and second tab channels, the tab hooks capturing the first and second tab extensions in the first and second tab channels to prevent inward movement of the first and second tab extensions.

19. The pedicle screw assembly of claim 16, wherein the ring body includes support pads between the first and second tab channels, the support pads engaging edges of the first and second tab extensions to prevent inward movement of the first and second tab extensions.

20. A pedicle screw assembly comprising:
a screw body having a head and a threaded shaft extending from the head;
a tulip having a pocket receiving the head of the screw body, the tulip having a first side wall and a second side wall on opposite sides of the pocket, the tulip having rod openings defined between the first and second side walls configured to receive a rod;
a set screw received in the pocket to secure the rod in the slot;
a tulip extension extending from the tulip, the tulip extension including a first extension tab extending from the first side wall and a second extension tab extending from the second side wall, the first and second side walls extending parallel to each other between proximal ends and distal ends of the first and second extension tabs, the tulip extension having a lumen defined between the first and second extensions, the tulip extension having slots defined between the first and second extensions aligned with the rod openings, the tulip extension including a deflectable retention spring along the first extension tab; and
a support ring including a ring body surrounding an opening configured to receive the distal ends of the first and second extension tabs, the ring body surrounding the first and second extension tabs to prevent opening of the first and second extension tabs relative to each other, the ring body coupled to the first and second extension tabs to support the first and second extension tabs and prevent closing of the first and second extension tabs relative to each other;
wherein the support ring interfaces with the retention spring to retain the support ring on the first and second extension tabs, and wherein the retention spring is releasable from the support ring to allow removal of the support ring from the distal ends of the first and second extension tabs.
